(19) United States
(12) Patent Application Publication
(10) Pub. No.: US 2024/0166671 A1
(43) Pub. Date: May 23, 2024

(54) Novel Stannic Protoporfin Compositions, Methods of Making, and Uses Thereof

(71) Applicant: Renibus Therapeutics, Inc., Southlake, TX (US)

(72) Inventor: Donald Jeffrey Keyser, Southlake, TX (US)

(21) Appl. No.: 18/363,548

(22) Filed: Aug. 1, 2023

Related U.S. Application Data
(60) Provisional application No. 63/394,146, filed on Aug. 1, 2022.

(51) Int. Cl. C07F 7/22; A61K 31/555; A61K 33/26; A61P 41/00

(52) U.S. Cl. CPC C07F 7/2284; A61K 31/555; A61K 33/26; A61P 41/00; C07F 7/2296

(57) ABSTRACT

Novel Stannic protoporfin compositions exhibiting high purity and other characteristics, including a novel solubility profile and visual indicators suggesting increase pharmacological activity, including enhanced antiviral activity. Also disclosed are novel processes for making Stannic protoporfin according to a process that requires fewer steps than known processes.

This application claims priority to U.S. Provisional Application No. 63/394,146 filed Aug. 1, 2022, the disclosure of which is hereby incorporated by reference in its entirety.

### BACKGROUND

Stannic protoporfin (Sn-protoporphyrin or SnPP) is known agent that undergoes proximal tubule uptake where it activates redox sensitive transcription factors, leading to the up-regulation of redox sensitive cytoprotective proteins. However, known methods of making SnPP includes a complicated process involving four distinct intermediate compounds and is known to have problems with stability and purity. SnPP is disclosed in U.S. Pat. No. 10,639,321 B2.

SnPP is also known to have some antiviral effect for particular kinds of viruses as disclosed by Neris et al., “Co-protoporphyrin IX and Sn-protoporphyrin IX inactivate Zika, Chikungunya and other arboviruses by targeting the viral envelope,” Sci Rep. 2018 Jun. 28; 8(1):9805. doi: 10.1038/s41598-018-27855-7. Neris et al. disclose that SnPP can be photosensitized and that non-photosensitized versions of SnPP may be used in photodynamic therapy for microorganism killing.

Accordingly, novel SnPP compositions having unique photodynamic properties are desired and that can be manufactured using convenient methods are needed.

### SUMMARY OF THE INVENTION

The present invention involves a stannic protoporfin composition that includes a compound of Formula (I):

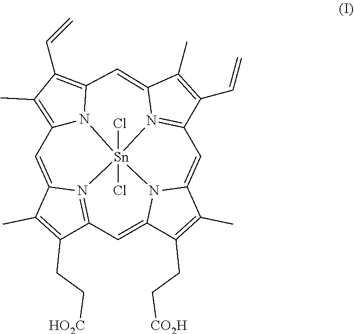

having a total level of impurities of 1.5% or less, as measured by gas chromatography. In an embodiment the level of impurities is 1% or less. In other embodiments the level of impurities is between 0.4 to 1%.

This composition may be preferably made from a process that includes two major steps. The process may include the following detailed process for making an protoporphyrin (IX) intermediate. For example, the method may include steps of (a) dissolving hemin in hot formic acid to form a first intermediate composition; (b) adding iron powder to the first intermediate composition to form a second intermediate composition; (c) adding a filtrate of the second intermediate composition to an aqueous solution of NH4OAc to precipitate a third intermediate composition; (d) dissolving the third intermediate composition in pyridine at an elevated temperature to form a fourth intermediate composition; (e) filtering the fourth intermediate composition to form a filtrate; and (f) precipitating a first intermediate compound according to Formula II:

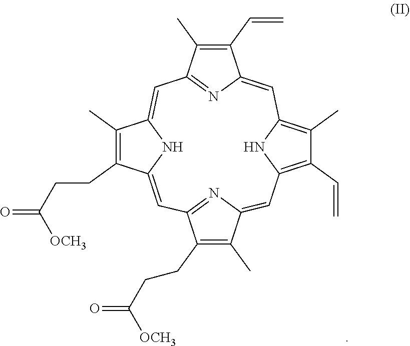 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

In another aspect, the method includes additions steps of adding the compound of Formula (II) to a mixture of stannous chloride, pyridine and glacial acetic acid to make a stannic protoporfin according to Formula I:

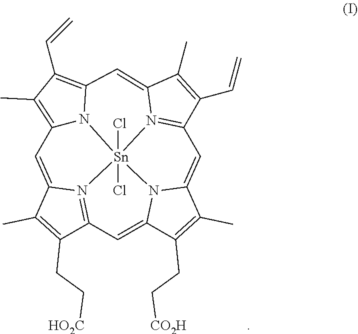

In another aspect, the present invention includes compositions of stannic protoporfin having a total level of impurities of 1.5% or less, as measured by gas chromatography an iron sucrose bicarb.

In another aspect, the SnPP having a total level of impurities of 1.5% or less is combined with iron sucrose and administered to a patient scheduled to undergo cardiothoracic surgery.

### BRIEF DESCRIPTION OF THE DRAWINGS

FIG. 1A shows the first step in the production of SnPP according to a comparative example.

FIG. 1B shows the second step in the production of SnPP according to the comparative example.

FIG. 1C shows the third step in the production of SnPP according to the comparative example.

FIG. 1D shows the fourth step in the production of SnPP according to the comparative example.

FIG. 2A shows the first step in the production of SnPP according to an example according to an embodiment of the invention.

FIG. 2B shows the second step in the production of SnPP according to an example according to an embodiment of the invention.

FIG. 3 shows a comparison of the impurity profiles present in the product of Example 1 compared to the Comparative Example.

FIG. 4 shows % cell viability after exposure to SnPP for Example 1 compared to the Comparative Example.

### DETAILED DESCRIPTION

The present invention relates to novel compositions of stannic protoporfin made according to a novel process that reduces the number of steps required relative to processes used to produce known stannic protoporfin compositions, and that exhibit an improved impurity profile and pharmacological properties relative to known Stannic protoporfin compositions.

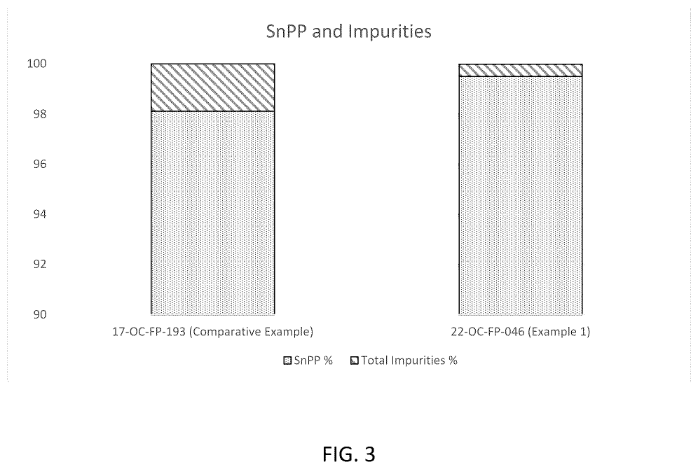

Disclosed are protoporphyrin compositions having a level of total impurities as measured by chromatography below 1.5%, more preferably below 1%, 0.9%, 0.8%, 0.7%, 0.6%, 0.5%, 0.4%, including compositions having a total impurity profile within a range of 0.1 to 1%, 0.2 to 1%, 0.3 to 1%, 0.4 to 1%, 0.5 to 1%, 0.6 to 1%, 0.7 to 1%. The chromatography method includes, for example, the chromatography method to produce the experimental results in Table I and FIG. 3.

SnPP composition according to an embodiment of the invention includes a compound of Formula (I):

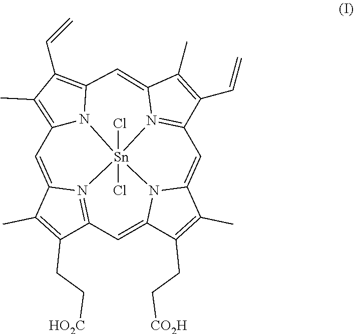

having a total level of impurities of 1.5% or less, as measured by gas chromatography. In an embodiment the level of impurities is 1% or less. In other embodiments the level of impurities is between 0.4 to 1%.

This composition may be preferably made from a process that includes two major steps. The process may include the following detailed process for making an protoporphyrin (IX) intermediate. For example, the method may include steps of (a) dissolving hemin in hot formic acid to form a first intermediate composition; (b) adding iron powder to the first intermediate composition to form a second intermediate composition; (c) adding a filtrate of the second intermediate composition to an aqueous solution of NH4OAc to precipitate a third intermediate composition; (d) dissolving the third intermediate composition in pyridine at an elevated temperature to form a fourth intermediate composition; (e) filtering the fourth intermediate composition to form a filtrate; and (f) precipitating a first intermediate compound according to Formula II:

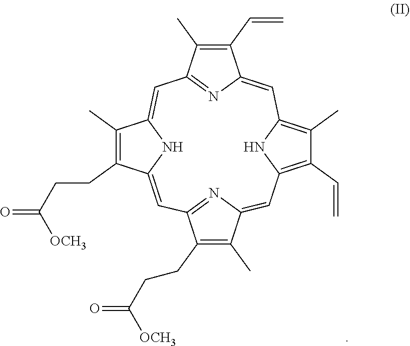 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

In another aspect, the method includes additions steps of adding the compound of Formula (II) to a mixture of stannous chloride, pyridine and glacial acetic acid to make a Stannic protoporfin according to Formula I:

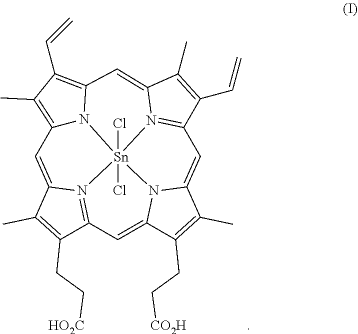

In another aspect, the SnPP with lower impurity profile is combined with iron sucrose and administered to a patient scheduled to undergo cardiothoracic surgery. The iron sucrose is preferably an iron sucrose composition that can be easily combined with tin protoporphyrin (SnPP) to form a stable solution that can be administered to a patient, as described in U.S. Pat. No. 11,292,813 filed Apr. 5, 2022, entitled “NOVEL IRON COMPOSITIONS AND METHODS OF MAKING AND USING THE SAME,” the subject matter of which is incorporated herein by reference. The composition is an aqueous iron pharmaceutical composition comprising: iron sucrose; bicarbonate; and a pharmaceutically acceptable aqueous carrier, wherein the iron sucrose is present in pharmaceutically effective amount for providing a protective effect to a patient's organs the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons.

The iron sucrose composition can be prepared, for example, by dissolving enough iron sucrose complex in water (ca 3.5 L) to give a 12 mg/mL (expressed as iron) solution when diluted to 6.0 L. The amount of iron sucrose needed was calculated for the final volume of liquid, 6100 mL (6.1 L) so that the final concentration is 12 mg/mL. This requires 73.2 g of iron. The use potency of iron sucrose is 0.0550. Thus, 73.2 g/0.0550 or 1331 g±1 g of iron sucrose is needed. Iron sucrose, 1331 g±1 g, was weighed directly into a 6.0 L Erlenmeyer flask. Approximately 3-3.5 L of water is added to the Erlenmeyer flask, and the contents of the flask are stirred.

Sodium bicarbonate is added in an amount such that the final sodium bicarbonate concentration is 10 mg/mL when diluted to 6.0 L. Sodium bicarbonate, 109.8±0.1 g, is weighed and added to the 6.0 L flask. Sodium chloride is added in an amount such that the final sodium chloride concentration is 9.0 mg/mL upon dilution. Sodium chloride, 54.9±0.1 g, is weighed and added to the 6.0 L flask. The suspension is stirred for 30-120 minutes to give a black opaque solution. The pH of the solution is monitored with a pH meter while 1 M sodium hydroxide is added in small portions until pH 10.30 is reached and remains stable. Sodium hydroxide, 40.0±0.1 g, was added to a 1.0 L Erlenmeyer flask. 1.0±0.1 L of water is added to the 1.0 L Erlenmeyer flask and stirred until all of the sodium hydroxide dissolved. A pH probe is affixed to monitor the pH of the 6.0 L Erlenmeyer flask and the sodium hydroxide is added in <100 mL portions until the pH=10.3±0.1. The solution is then stirred for 10 minutes. The pH is checked again after 10 minutes and if necessary adjusted to within pH=10.3±0.1.

The solution is then transferred to a volumetrically accurate flask and diluted to 6.1 L with water. A 2 L volumetric flask is used twice to transfer exactly 4 L of the 10.3 pH solution to a 6 L Erlenmeyer flask. The remaining 10.3 pH solution is diluted to 2 L in a volumetric flask and added to the 6 L Erlenmeyer flask. The 100 mL graduated cylinder is used to add 100±0.1 mL to the 6.0 L Erlenmeyer, and the resulting solution is stirred for 10 minutes.

The resulting product solution appears dark red to brown. Two isotopes of iron are present in the sample preparation in a ratio consistent with that of the standard preparation. The resulting material had a pH of 10.3, which is within the preferred limits of 10.1-10.4. The resultant material had 11.5/11.6 parts per thousand (mg/mL) iron according to SOP 174472, which determines iron through inductively coupled plasma-mass spectroscopy.

The iron sucrose made according to the above-noted process has been found to exhibit an elevated induction of renal heme oxygenase (HO-1). Specifically, intravenous administration of the iron sucrose (FeS) bicarb made according to the above process resulted in elevated renal heme oxygenase (HO-1) after four hours relative to commercially available iron sucrose (FeS) composition sold under the brand name, Venofer®. The results are shown in Table 2 below:

TABLE 2
Kidney mRNA HO-1/GAPDH

| Run# | Control | 4 hr IV FeS, Venofer(R) | 4 hr IV FeS-bicarb |
|---|---|---|---|
| 1 | 0.22 | 1.52 | 3.2 |
| 2 | 0.04 | 1.23 | 2.01 |
| 3 | 0.06 | 1.11 | 1.99 |
| 4 | 0.07 | 2.23 | 2.23 |
| 5 | | 1.86 | 1.86 |
| Avg. | 0.1 | 1.59 | 2.34 |
| Std. Err | 0.04 | 0.21 | 0.23 |

The treatment method involves combining the SnPP and FeS in a bolus, and the combined composition is administered to the patient in a single intravenous infusion dose.

### Comparative Example

A first composition of Stannic protoporfin was made a comparative example as follows.

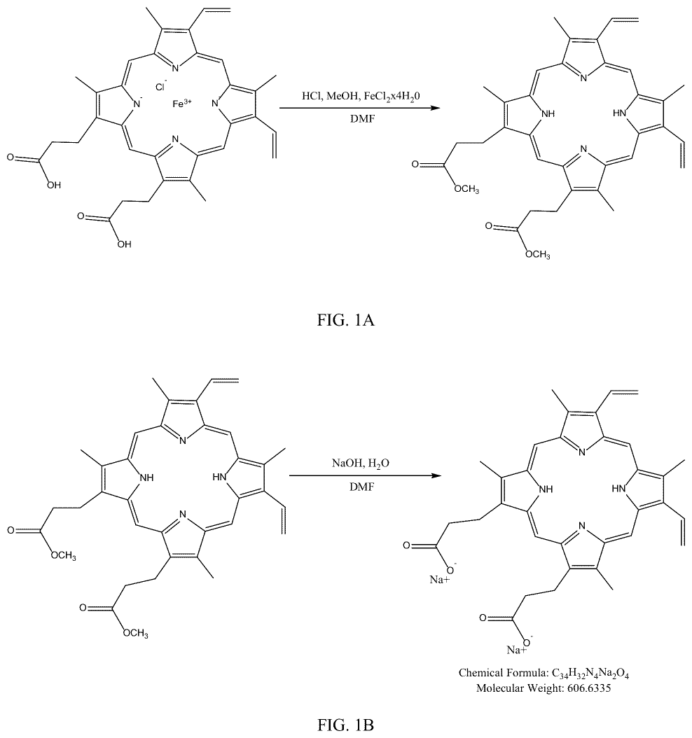

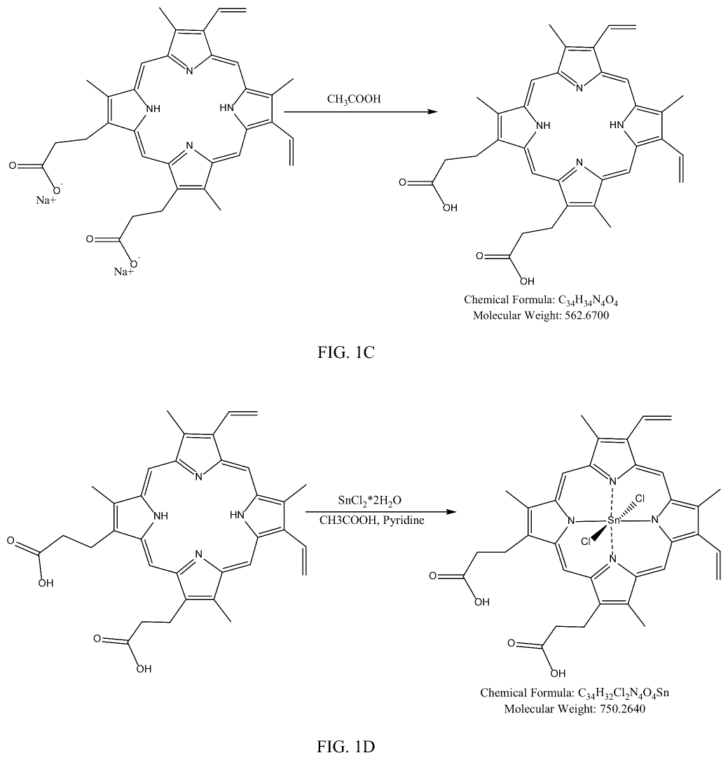

Hemin was dissolved in dimethylformamide at 50-60° C. and filtered. In a separate vessel methanol was saturated with gaseous HCl such that it was heated to 45° C. multiple times. Iron (II) chloride tetrahydrate was then added to the methanol/HCl solution. At this point, the filtered Hemin solution was added by peristaltic pump at ˜25 ml/min such that the temperature was maintained between 40-60° C. Following complete addition of the hemin solution, the reaction was stirred under HCl gas for another 30 minutes. The reaction mixture was then diluted with dichloromethane and washed with water. Aqueous washes are then back-extracted with dichloromethane. The organics were combined and washed once more with water. Following separation, the organics were then split into two portions and purified by silica gel chromatography using a dichloromethane, ethyl acetate gradient. Product fractions were then combined and stripped of solvent by rotary evaporation until ˜25% of the original volume was reached. Ethyl acetate was then added and rotary evaporation was continued and the final slurry was then filtered to isolate a dark purple solid which is protoporphyrin IX dimethyl ester as shown in FIG. 1A.

Protoporphyrin IX dimethyl ester was dissolved in dimethylformamide at 105° C. under inert atmosphere. Aqueous NaOH was added and the temperature was increased to 110° C. The reaction was stirred for 3 hours and allowed to cool. The following day, the reaction was cooled to <6° C. and filtered. The crude protoporphyrin IX disodium salt was washed with cold DMF and acetone. After the product has sufficiently dried on the filter, it was ground with a mortar and pestle and dried in a vacuum over at 80° C. overnight. This step is illustrated in FIG. 1B.

Protoporphyrin IX disodium salt was dissolved in glacial acetic acid under inert atmosphere and stirred at room temperature for at least 24 hours. The reaction was filtered to isolate a red/brown solid. The product was then suspended in 0.5 N acetic acid, stirred overnight and filtered again. Depending on purity of the protoporphyrin IX, one or more recrystallizations was carried out using pyridine at 80° C. followed by cooling to −20° C. and filtration as shown in FIG. 1C.

Glacial acetic acid and pyridine were charged into a 22 L reaction vessel with inert atmosphere at 50° C. Stannous chloride dihydrate was added and stirred to complete dissolution. Protoporphyrin IX was then added and stirred at temperature for a minimum of 48 hours. The resulting solution was then cooled to room temperature, filtered, and rinsed with glacial acetic acid. The final product was then vacuum dried and slurried in hydrochloric acid followed by filtration, deionized water rinse, and finally vacuum drying. This step is shown in FIG. 1D.

### Example 1

A first composition of Stannic protoporfin was made using a two-step process as follows:

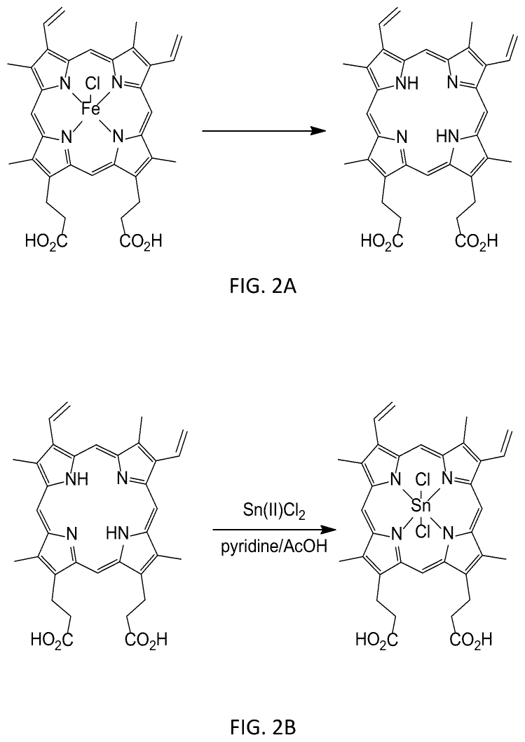

Hemin was dissolved in hot formic acid, then iron powder was added in aliquots over 20 min. The resulting mixture was heated and stirred for 30 min, then filtered through Celite. The filtrate was added to a stirring aqueous solution of NH₄OAc to precipitate the desired product, which was filtered and dried. This crude material was dissolved in hot pyridine and the hot solution was filtered through Celite. The purified product precipitated from the filtrate upon cooing and was recovered by filtration. This corresponds to the first step in this process as shown in FIG. 2A.

Stannous chloride was dissolved in pyridine under inert atmosphere, glacial acetic acid was added and the mixture is heated at 50° C. Protoporphyrin IX was then added and stirred and heated for a minimum of 24 hours and monitored for completion by HPLC. The reaction was cooled to room temperature and filtered. The product was then triturated first with water, then 2 M HCl (aq) and then again with water. An IPC was conducted to determine if a pyridine/AcOH recrystallization, followed by an additional water trituration. The product was then dried to remove residual solvents. This corresponds to the second step in this process as shown in FIG. 2B.

The level of impurities in the Stannic protoporfin was then determined by chromatography. The results are shown in Table I below:

TABLE I
Stannic protoporfin Impurity Profiles

| Batch | Peak | Area % | Total Impurities % |
|---|---|---|---|
| 17-OC-FP-193 | 1 | 0.121 | 1.891 |
| (Comparative | 2 | 0.041 | |
| Example) | 3 | 0.637 | |
| | 4 | 0.678 | |
| | 5 | 0.336 | |
| | SnPP | 98.11 | |
| | 7 | 0.078 | |
| 22-OC-FP-046 | 1 | 0.12 | 0.48 |
| (Example 1) | 2 | 0.06 | |
| | 3 | 0.11 | |
| | SnPP | 99.5 | |
| | PPIX | 0.19 | |

The Stannic protoporfin composition of Example 1 had an almost 4-fold drop in total percent impurities relative to the comparative example. The inventors believe based on preliminary solubility data and other observations, including for example visually observed color, that the composition of Example 1 will likely exhibit unique photodynamic properties, including for example enhanced biological activity, such as enhanced antiviral activity.

### Example 2

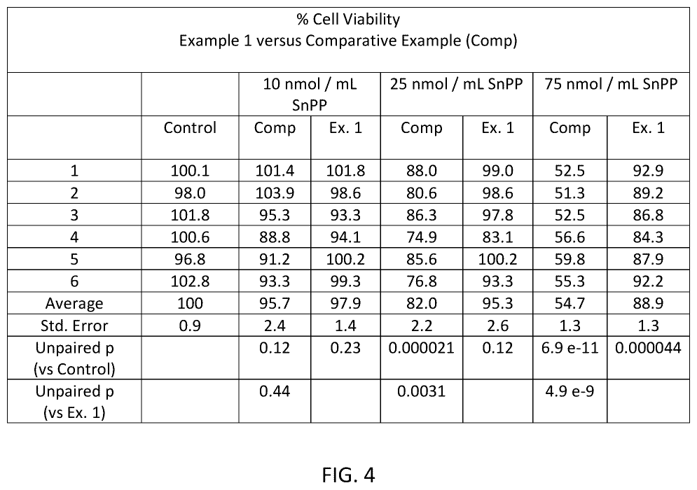

The composition of Example 1 and the Comparative Example were exposed to incubated cultured human tubule cells with different concentrations of SnPP. These data are summarized in Table 3 below. Detailed % viability data is shown in FIG. 4.

TABLE 3
% Viable Cells, n = 6, control 100%

| SnPP (nmol/mL) | Comparative Example | Example 1 |
|---|---|---|
| 75 | 54.7 | 88.9 |
| 25 | 82.0 | 95.3 |
| 10 | 95.7 | 97.9 |

At the highest concentration of SnPP, the amount of cell viability observed for the composition of Example 1 was 88.9% (11% killing) compared to 54.7% cell viability (46% cell killing) for the Comparative Example. These data show that the SnPP composition of Example 1 results in less cell death than the composition made according to the comparative example.

### Example 3

The SnPP composition of Example 1 and the Comparative Example (1 micromole) were combined with iron sucrose (1 mg) to assess biomarkers for oxidative stress. The results are shown in Tables 4A-4B:

TABLE 4A
HO-1/GAPDH mRNA (Cortex)

| | | 4 hr IV FeS + | 4 hr IV FeS + |
|---|---|---|---|
| | Normal | SnPP(Ex1) | SnPP(Comp) |
| 1 | 0.2 | 5.2 | 14.3 |
| 2 | 0.1 | 14.3 | 10.0 |
| 3 | 0.2 | 9.8 | 19.9 |
| Avg. | 0.18 | 9.76 | 14.70 |
| Std. Err | 0.03 | 2.62 | 2.87 |
| unpaired P | | 0.022 | 0.0072 |
| (vs normal) | | | |
| unpaired P | | | 0.27 |
| (vs Ex. 1) | | | |

TABLE 4B
HO-1/GAPDH mRNA (Log 10)

| | | 4 hr IV FeS + | 4 hr IV FeS + |
|---|---|---|---|
| | Normal | SnPP(Ex1) | SnPP(Comp) |
| 1 | −0.78 | 0.7 | 1.2 |
| 2 | −0.9 | 1.2 | 1.0 |
| 3 | −0.6 | 1.0 | 1.3 |
| Avg. | −0.76 | 0.95 | 1.15 |
| Std. Err | 0.07 | 0.13 | 0.09 |
| unpaired P | | 0.00032 | 0.000074 |
| (vs normal) | | | |
| unpaired P | | | 0.27 |
| (vs Ex. 1) | | | |

These data show that the composition of Example 1 exhibits a lower HO-1 induction, which is an indicator of oxidative stress, relative to the composition of the comparative example. These data considered in light of that in Example 2 demonstrate that the SnPP produced according to the method of Example 1, exhibit a unique biomarker profile that suggests a therapeutic use can be achieved with an improved safety profile.

### Example 4

The SnPP produced according to Example 1 was administered with iron sucrose to patients scheduled to undergo cardiothoracic surgery according to the process described in U.S. Provisional Patent Application No. 63/499,974, entitled “Method of Protecting an Organ Prior to Surgery,” filed on May 3, 2023. This method involved combining 90 mg of SnPP and 240 mg of FeS-bicarb.

### Example 5

The SnPP produced according to Example 1 was administered with iron sucrose to patients scheduled to undergo cardiothoracic surgery according to the process described in U.S. Provisional Patent Application No. 63/499,974, entitled “Method of Protecting an Organ Prior to Surgery,” filed on May 3, 2023. This method involved combining 45 mg of SnPP and 240 mg of FeS-bicarb.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A stannic protoporfin composition comprising a compound of Formula (I) 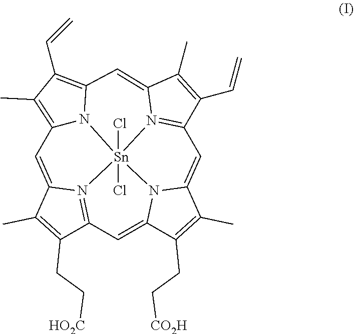
wherein the composition comprises a total level of impurities of 1.5% or less, as measured by gas chromatography.

2. The composition of claim 1, wherein the total level of impurities is 1% or less.

3. The composition of claim 1, wherein the total level of impurities in the range of 0.1 to 1%.

4. The composition of claim 1, wherein the total level of impurities in the range of 0.4 to 1% or less.

5. A method of making Stannic protoporfin comprising the steps of:
(a) dissolving hemin in hot formic acid to form a first intermediate composition;
(b) adding iron powder to the first intermediate composition to form a second intermediate composition;
(c) adding a filtrate of the second intermediate composition to an aqueous solution of NH₄OAc to precipitate a third intermediate composition;
(d) dissolving the third intermediate composition in pyridine at an elevated temperature to form a fourth intermediate composition;
(e) filtering the fourth intermediate composition to form a filtrate; and
(f) precipitating a first intermediate compound according to Formula II:

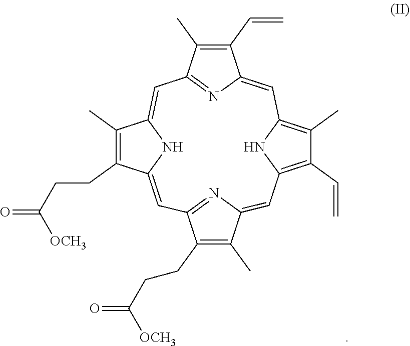 [SMILES: C=CC1=C(C)c2cc3[nH]c(cc4nc(cc5[nH]c(cc1n2)c(C)c5C=C)C(C)=C4CCC(=O)OC)c(CCC(=O)OC)c3C]

6. The method of claim 6, further comprising adding the compound of Formula (II) to a mixture of stannous chloride, pyridine and glacial acetic acid to make a stannic protoporfin according to Formula I:

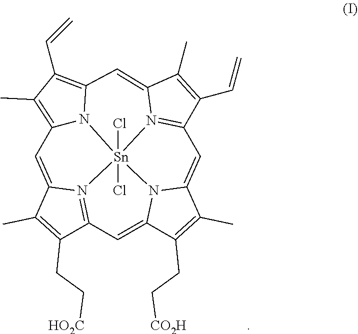

7. The method of claim 6, wherein adding the compound of Formula (II) to a mixture of stannous chloride, pyridine and glacial acetic acid is conducted in an inert environment.

8. A stannic protoporfin composition made according to the process of claim 7.

9. A pharmaceutical composition comprising the stannic protoporfin composition of claim 1 and an iron pharmaceutical composition comprising: iron sucrose; bicarbonate; and a pharmaceutically acceptable aqueous carrier, wherein the iron sucrose is present in pharmaceutically effective amount, the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons.

10. A pharmaceutical composition comprising the stannic protoporfin composition of claim 8 and an iron pharmaceutical composition comprising: iron sucrose; bicarbonate; and a pharmaceutically acceptable aqueous carrier, wherein the iron sucrose is present in pharmaceutically effective amount, the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons.

11. A method of reducing post operative complications of a human patient from injury based on a scheduled or anticipated insult comprising administering to the patient the composition of claim 9.

12. A method of reducing post operative complications of a human patient from injury based on a scheduled or anticipated insult comprising administering to the patient the composition of claim 10.
